(19) United States
(12) Patent Application Publication
(10) Pub. No.: US 2017/0334824 A1
(43) Pub. Date: Nov. 23, 2017

(54) METHOD FOR EXTRACTING AROMATIC PRODUCTS OF VALUE FROM COMPOSITIONS CONTAINING LIGNIN

(71) Applicant: BASF SE, Ludwigshafen (DE)

(72) Inventors: Ralf PELZER, Fürstenberg (DE); Carolin REGENBRECHT, Mannheim (DE); Chung Huan WONG, Mannheim (DE); Gabriele IFFLAND, Heidelberg (DE); Agnes VOITL, Schifferstadt (DE)

(73) Assignee: BASF SE, Ludwigshafen (DE)

(21) Appl. No.: 15/522,550

(22) PCT Filed: Nov. 6, 2015
(86) PCT No.: PCT/EP2015/075869

§ 371 Date: Apr. 27, 2017

(30) Foreign Application Priority Data
Nov. 7, 2014 (EP) 14192283.1

(51) Int. Cl. C07C 45/79; C07C 47/58

(52) U.S. Cl. CPC C07C 45/79; C07C 47/58

(57) ABSTRACT

The present invention relates to a method for extracting aromatic compounds from aqueous, alkaline compositions containing lignin, which have a pH value of at least pH 10, in particular of at least pH 11, especially of at least pH 12, characterized in that the aqueous, alkaline composition containing lignin is treated with active carbon, the active carbon separates from the aqueous, alkaline composition containing lignin and the active carbon then undergoes a desorption step in order to extract the aromatic compounds, wherein the desorption step comprises the treatment of the active carbon
(i) by means of an organic solvent, which essentially consists of one or more aromatic hydrocarbons or a mixture of at least one aromatic hydrocarbon together with at least one C₁-C₄ alkanol, or
(ii) by means of water vapor,
   wherein an eluate is obtained which contains the aromatic compounds.

The present invention relates to a process for obtaining aromatic valuable products from aqueous alkaline lignin-comprising compositions.

### BACKGROUND OF THE INVENTION

The transformation of renewable raw materials to valuable chemicals which are suitable, in particular, as fragrances and flavorings, is of very great interest. The biopolymer lignin which is incorporated in the cell wall of the plant cells during lignifications forms 20 to 30% of the dry mass of lignified plants. In the processing of wood to wood pulp, therefore, large amounts of lignin and also lignin-comprising substances, such as alkali lignin, lignin sulfate or ligninsulfonate, as waste materials or by products arise. The total production of lignin-comprising substances is estimated at about 20 billion tons per year. Some of the lignin that arises in wood processing is further used at this time. For example, alkali lignin which is producible by alkaline treatment of the black liquor arising in paper manufacture, is used in North America as a binder for wood- and cellulose-based pressed sheets, as dispersant, for clarification of sugar solutions, for stabilization of asphalt emulsions and also for foam stabilization. However, by far the greatest part of waste lignin is used by combustion as an energy source, e.g. for the pulp process.

The biopolymer lignin comprises a group of three-dimensional macromolecules which occur in the cell wall of plants and which are composed of differing phenolic monomer building blocks such as p-coumaryl alcohol, coniferyl alcohol and sinapyl alcohol. On account of its composition, in addition to petroleum, it is the single significant source of aromatics in nature. Lignin is therefore a valuable renewable material. The utilization of this renewable natural material, in addition, does not compete with a use as food.

In the prior art, obtaining individual aromatic valuable materials such as, for example, 4-hydroxy-3-methoxybenzaldehyde (vanillin) from lignin-comprising compositions is known.

EP 2157184 A1 DE 2928862 A1 describes a process involving biotransformation for extracting vanillin from a ferulic acid, in which the solution obtained after the biotransformation which comprises vanillin, ferulic acid, vanillic acid, vanillyl alcohol and guaiacol is treated with activated carbon or a synthetic resin with the aim of extracting the vanillin. The vanillin that is bound to the activated carbon or synthetic resin is then desorbed using 95% strength ethanol. The pH of the solution obtained after the biotransformation is in the range from 7 to 9.

DE 2928862 A1 describes a process for utilizing the sulphide waste liquor arising when wood pulp is extracted industrially, in which the sulphide waste liquor is admissed with phosphoric acid and heated, wherein, in addition to sulfur-dioxide, other volatile products, in particular formic add, acetic acid and the aromatics furfural and cymene are also expelled from the sulphide waste liquor by introducing air. The majority of the sulfur dioxide, formic acid and acetic acid present in the vapor are condensed. The non-condensed gaseous components (off-gas) are passed through milk of lime, a calcium sulfite solution, or a CaCO₃ suspension and the remaining residue of non-condensed off-gas which comprises residual SO₂ and also, in particular, furfural and cymene, is taken up by activated carbon. The components taken up by activated carbon can then be recovered by heating, for example by means of steam. This process is suitable at best with limitations for extracting aromatic materials of value from lignin-comprising compositions, since said process comprises many process steps, comprises a gas extraction, and generates many waste materials that need to be disposed of. In addition, large amounts of mineral acids are required.

M. Zabková et al., Sep. Purif. Technol. 2007, 55, 56-68 describe the extraction of aromatic valuable materials, in particular of vanillin or vanillate, from alkaline lignin solutions via cation exchange, with neutralization of the alkali solution. In this case the vanillate is through a cation exchanger in the H⁺ form, whereby it is protoniated to form vanillin. This cation exchange is coupled to a neutralization in the presence of a buffer solution (vanillate/vanillin). This process requires large amounts of acid for neutralization of the alkaline reaction medium. By acidification, the lignin precipitates out of the solution, must be filtered off and can lead to a loss due to filtration of the desired aromatic valuable materials.

M. Zabková et al., J. Membr. Sci. 2007, 301 (1-2), 221-237 in addition describes obtaining from the aqueous alkaline reaction streams arising in the oxidation of craft lignin using ultrafiltration through tubular ceramic membranes. A disadvantage is the comparatively high expenditure of ultrafiltration and the associated costs, and also the low load capacity. Thus, efficient removal of the vanillin is only possible at low permeation rates. Membranes that permit higher permeation rates lead to an increased discharge of the lignin, the removal of which requires further separation steps. Furthermore, the oxidic membrane structures are unsuitable for a long exposure to alkaline medium, since they are subject to corrosion.

WO 2014/006108 A1 describes a process for obtaining vanillin from aqueous alkaline vanillin-containing compositions, as arise, for example, in the oxidation of aqueous alkali lignin-comprising solutions or suspensions in which the alkaline vanillin-containing compositions are treated with an anion exchanger resin. The vanillin that is bound to the anion exchanger resin is desorbed, in addition to further bound aromatic valuable materials, using dilute mineral acids in methanol or by means of acetic acid in ethyl acetate.

In this process also, acids are required for desorption of the aromatic valuable materials, for example vanillin, that are bound to the anion exchanger resin. A disadvantage of obtaining aromatic valuable products using ion-exchange resins is, in addition, that this is a chemical operation and the products obtained thereby can no longer be classified as “natural”. Furthermore, the ion exchangers, in the adsorption of aromatic compounds, do not have sufficient selectivity and efficacy, since inorganic materials present in the alkali lignin-comprising solutions are also bound and thereby a large part of the adsorption capacity of the ion exchangers is lost.

### SUMMARY OF THE INVENTION

The object of the present invention is to provide a robust and efficient process for obtaining aromatic valuable materials from aqueous alkaline lignin-comprising compositions, in which the use of large amounts of acid can substantially be avoided. The process in addition shall be based as far as possible on a purely physical process and permit the aromatic valuable materials to be obtained in good selectivity from the aqueous alkaline lignin-comprising compositions.

These and other objects are achieved by the process described hereinafter for obtaining aromatic compounds from aqueous alkaline lignin-comprising compositions that have a pH of at least pH 10, in particular at least pH 11, especially at least pH 12. The method comprises treating an aqueous alkaline lignin-comprising composition with activated carbon, wherein the aromatic compounds present in the aqueous alkaline lignin-comprising compositions are physically adsorbed to the surface of the activated carbon. Subsequent desorption with an organic solvent which substantially consists of one or more aromatic hydrocarbons, or is a mixture of at least one aromatic hydrocarbon with at least one C₁-C₄ alkanol, and/or with steam yields an eluate which comprises the aromatic compounds.

The present invention therefore relates to a process for obtaining aromatic compounds from aqueous alkaline lignin-comprising compositions that have a pH of at least pH 10, in particular at least pH 11, especially at least pH 12, which comprises treating the aqueous alkaline lignin-comprising composition with activated carbon, separating off the activated carbon from the aqueous alkaline lignin-comprising composition and then subjecting the activated carbon to a desorption step for obtaining the aromatic compounds, wherein the desorption step comprises treating the activated carbon
(i) with an organic solvent which substantially consists of one or more aromatic hydrocarbons, or is a mixture of at least one aromatic hydrocarbon with at least one C₁-C₄ alkanol or
(ii) with steam, wherein an eluate that comprises the aromatic compounds is obtained.

The process according to the invention is distinguished, in particular, in that, for obtaining the aromatic valuable materials from aqueous alkaline lignin-comprising compositions, the use of acids can substantially be avoided. The valuable materials desorbed from the activated carbon comprise virtually no salts and may therefore be fed without workup to further purification steps.

The aromatic valuable materials present in the aqueous alkaline lignin-comprising compositions will adsorb on the activated carbon used in the present process and thereby permit the removal thereof in good selectivities. The valuable materials bound to the activated carbon in addition may be desorbed readily, as a result of which they can be recovered in high yields. The process therefore permits an efficient and continuous removal of aromatic valuable materials formed during the breakdown of lignin.

The process according to the invention is simple and robust and may also be carried out on an industrial scale.

The present process in addition is based on a purely physical process. The valuable products that are obtained from alkaline lignin-comprising compositions using this process which are formed in the natural oxidative breakdown of lignin can therefore be classified as “natural”.

### DETAILED DESCRIPTION OF THE INVENTION

In principle, any desired aqueous lignin-comprising compositions can be used in the process according to the invention that have an alkaline pH, wherein the pH is generally at least pH 10, in particular at least pH 11, and especially at least pH 12, and can also be pH 14.

Generally, in the process according to the invention, an aqueous alkaline lignin-comprising composition can also be used which has previously been treated with alkalis or oxidatively. In this case, in the context of the present invention, this is an aqueous alkaline lignin-comprising composition which has been obtained by dissolving a lignin or lignin derivative in aqueous alkali and/or by partial oxidation, especially by electrolysis, of an aqueous alkaline lignin-comprising composition.

The lignin or lignin derivative used for producing the aqueous alkaline lignin-comprising composition is selected, for example, among lignin from black liquor, craft lignin, lignosulfate, lignosulfonate, alkali lignin, soda lignin, Organosolv lignin or corresponding residues which arise in an industrial process such as production of papermaking stock, wood pulp or cellulose, e.g. lignin from black liquor, from the sulfite process, from the sulfate process, from the Organocell or Organosolv process, from the ASAM process, from the craft process, or from the natural pulping process.

Correspondingly, the aqueous alkaline lignin-comprising composition used for the partial oxidation is an alkaline solution or suspension which arises as by-product in an industrial process such as the production of papermaking stock, wood pulp or cellulose, e.g. black liquor, and also the lignin-comprising wastewater streams from the sulfite process, from the sulfate process, from the Organocell or Organosolv process, from the ASAM process, from the craft process or from the natural pulping process.

The aqueous alkaline lignin-comprising composition that is optionally treated with alkalis or oxidatively generally has a pH of at least pH 10, frequently at least pH 11, in particular at least pH 12.

The aqueous lignin-comprising composition which has optionally been treated with alkalis or oxidatively generally comprises 0.5 to 30% by weight, preferably 1 to 15% by weight, in particular 1 to 10% by weight, of lignin, based on the total weight of the aqueous lignin-comprising composition.

In a preferred embodiment of the process according to the invention, as aqueous alkaline lignin-comprising composition, an aqueous lignin-comprising wastewater stream from the production of papermaking stock, wood pulp or cellulose is used.

In a particularly preferred embodiment of the process according to the invention, for production of the aqueous alkaline lignin-comprising composition, black liquor from the papermaking industry, papermaking stock production or cellulose production is used.

As alkalis or bases for producing the aqueous alkaline lignin-comprising compositions, or for setting the pH of the aqueous alkaline lignin-comprising compositions, primarily, inorganic bases can be used, e.g. alkali metal hydroxides such as NaOH or KOH, ammonium salts such as ammonium hydroxide, and alkali metal carbonates, such as sodium carbonate, e.g. in the form of soda. Preference is given to alkali metal hydroxides, in particular NaOH and KOH. The concentration of inorganic bases in the aqueous lignin-comprising suspension or solution should not exceed 5 mol/l, in particular 4 mol/l, and is typically in the range from 0.01 to 5 mol/l, in particular in the range from 0.1 to 4 mol/l.

In a first step of the process according to the invention, the aqueous alkaline lignin-comprising composition is treated with activated carbon and the activated carbon is then separated off from the aqueous alkaline lignin-comprising composition. In this case, the aromatic compounds are adsorbed on the activated carbon. This operation is also termed a loading step.

For the process according to the invention, generally any commercially available activated carbon can be used. Suitable activated carbons are primarily not activated carbons that are chemically activated, or can be chemically pretreated, e.g. base-impregnated or washed.

In a preferred embodiment of the process according to the invention, the activated carbon that has been activated is activated carbon that has been activated with steam. The activated carbon that has been activated with steam is generally commercially available activated carbons such as, for example, CAL® or Aquacarb® 207C from Chemviron Carbon, Norit® ROY 0.8 and Norit® GAC 1240 from Norit or Epibon® A 8×30 or Hydraffin® 30N from Donau Carbon.

The base-impregnated activated carbon is an activated carbon which has been pretreated with bases, as defined above. Preferably, the base-impregnated activated carbon is activated carbon which has been pretreated with NaOH. For the impregnation, the activated carbon that has been activated is generally washed more than once with an aqueous solution of the base.

Generally, the activated carbon used according to the invention has a specific surface area in the range from 500 to 1500 m²/g, preferably in the range from 700 to 1300 m²/g, determined by nitrogen adsorption by the BET method as specified in DIN ISO 9277:2003-05.

The activated carbon used according to the Invention in addition usually has an adsorption capacity of at least 15 g of methylene blue per 100 g of activated carbon, preferably of at least 20 g of methylene blue per 100 g of activated carbon.

The grain size of the activated carbon used according to the invention is usually in the range from 0.2 to 5 mm, preferably in the range from 0.4 to 3 mm.

The aqueous alkaline lignin-comprising composition is generally treated with the activated carbon at a temperature in the range from 10 to 100° C., preferably in the range from 10 to 70′C, in particular in the range from 15 to 50° C.

The aqueous alkaline lignin-comprising composition is generally treated with the activated carbon at ambient pressure, but can also be treated at elevated pressure, in particular when the activated carbon is present as a bed, or fixed bed, for example in the form of a packed column. Preferably, the aqueous alkaline lignin-comprising composition is treated with the activated carbon at a pressure in the range from 1 to 50 bar, preferably in the range from 1 to 30 bar, particularly preferably in the range from 1 to 10 bar.

To treat the aqueous alkaline lignin-comprising composition with activated carbon, said activated carbon can be added, for example, to the aqueous alkaline lignin-comprising composition. After a certain residence time, the activated carbon is separated off from the aqueous alkaline lignin-comprising composition. It can be separated off by usual processes of solid-liquid separation, e.g. by filtration, sedimentation or centrifugation.

Preferably, for loading the activated carbon, the aqueous alkaline lignin-comprising composition is passed once or more than once over at least one bed, or fixed bed, of activated carbon, for example through one or more parallel or sequentially arranged columns packed with activated carbon, hereinafter also termed adsorbent arrangement.

The aqueous alkaline lignin-comprising composition can be passed through the adsorbent arrangement not only downwardly, but also upwardly. Preferably, it is passed through downwardly. The specific flow rate (specific loading rate) is preferably in the range from 0.2 to 35 bed volumes per hour (BV/h), in particular in the range from 0.5 to 10 BV/h, especially in the range from 1 to 5 BV/h. The passage through proceeds preferably at a linear velocity in the range from 0.1 to 50 m/h.

The relative amount of lignin-comprising suspension or solution and solid activated carbon is usually selected in such a manner that at least 35%, and in particular at least 50%, of the aromatic valuable materials present in the aqueous alkaline composition are absorbed by the activated carbon. The amount of aqueous alkaline composition to 100 times the amount, in particular 2 to 50 times the amount of the bed volume. Depending on the degree of adsorption, the effluent arising at the outlet of the adsorbent arrangement, e.g. the column packed with adsorbent, can still comprise aromatic valuable materials, and so the effluent can optionally be passed to a further adsorbent arrangement, e.g. a further column packed with activated carbon.

Optionally, subsequently to the adsorption and separating of the activated carbon from the aqueous alkaline lignin-comprising composition, a washing step can proceed. Usually, for the washing of the activated carbon loaded with the aromatic compounds, an aqueous liquid is used. An aqueous liquid is taken to mean water or a mixture of water with a water-miscible organic solvent, wherein water comprises the main component of the mixture and in particular 90% by volume of the mixture. The pH of the aqueous liquid is usually in the neutral range, i.e. in the range from pH 6 to pH 8. The washing step generally proceeds at a temperature and a pressure as defined above for the loading of the activated carbon. If the activated carbon is loaded in an adsorbent arrangement, the aqueous liquid, in particular water, is passed through the adsorbent arrangement upwardly or downwardly. The amount of aqueous liquid, hereinafter also wash water, is at this stage usually 1 to 20 times the bed volume, in particular 2 to 10 times the bed volume. The passage of the wash water generally proceeds at a specific flow rate (specific loading) in the range from 0.5 to 10 BV/h, in particular in the range from 1 to 8 BV/h, or a linear velocity in the range from 0.1 to 50 m/h. The wash waters arising in this case can comprise small amounts of aromatic valuable materials, and can then be combined with the effluent arising during loading.

Optionally, subsequently to the loading step, or in particular subsequently to the wash step and before the desorption, the activated carbon can be treated with an aqueous solution of an acid, in particular of a mineral acid or an organic sulfonic acid. In this case, the aromatic valuable materials bound to the activated carbon are protonated or neutralized. Subsequently, the valuable materials are desorbed by treating the activated carbon either with an organic solvent which substantially consists of one or more aromatic hydrocarbons, or is a mixture of at least one aromatic hydrocarbon with at least one C₁-C₄ alkanol or with steam.

Suitable mineral acid hydrochloric acid, nitric acid, perchloric acid, phosphoric acid or sulfuric acid. Suitable organic sulfonic acids are, primarily, methanesulfonic acid. A particularly preferred mineral acid is sulfuric acid. Preferably, the aqueous solution of the acid has an acid concentration in the range from 0.01 to 10 mol kg⁻¹, preferably in the range from 0.1 to 5 mol kg⁻¹, in particular 0.1 to 2 mol kg⁻¹.

Optionally, the activated carbon is washed with water before and/or after the treatment with aqueous dilute acid.

If the activated carbon is loaded in an adsorbent arrangement, the aqueous dilute acid, optionally after a wash step, is passed upwardly or downwardly through the adsorbent arrangement in order to protonate any bound anionic aromatic valuable materials. The amount of aqueous dilute acid is usually 0.1 to 10 times the bed volume, in particular 0.5 to 5 times the bed volume. The aqueous dilute acid is generally passed through at a specific flow rate (specific loading rate) in the range from 0.5 to 10 BV/h, in particular in the range from 1 to 8 BV/h.

If a treatment with an aqueous dilute acid is carried out, this treatment can be followed by a further wash step with an aqueous liquid, in particular water. With respect to the amount of the aqueous liquid, and the flow rate, that stated in connection with the above described wash step applies.

Subsequently to the adsorption and the optionally followed wash step, the valuable materials bound to the activated carbon are liberated (desorption). For the desorption, the activated carbon is treated either with an organic solvent which substantially consists of one or more aromatic hydrocarbons, or is a mixture of at least one aromatic hydrocarbon with at least one C₁-C₄ alkanol (variant (i)) or with steam (variant (i)).

Generally suitable as aromatic hydrocarbons which can be used for the desorption of the aromatic valuable materials bound on the activated carbon, according to the first variant (i), are any aromatic hydrocarbons customarily used as solvent, and also mixtures thereof.

The aromatic hydrocarbons used in the process according to the invention for desorption of the aromatic valuable materials bound to the activated carbon according to the first variant (i) are, for example, non halogenated aromatic hydrocarbons, such as benzene, toluene or xylenes, and halogenated aromatic hydrocarbons, such as chlorobenzene or dichlorobenzenes and mixtures thereof. Preferably, the aromatic hydrocarbons are hydrocarbons, toluene or xylenes and mixtures thereof.

The organic solvent used for the desorption in the first variant (i) in general comprises at least 80% by weight, preferably at least 90% by weight, particularly preferably at least 95% by weight or more, for example up to 97% by weight, of one or more aromatic hydrocarbons, as defined above, or a mixture of at least one aromatic hydrocarbon, as defined above, with at least one C₁-C₄ alkanol. If a mixture of at least one aromatic hydrocarbon as defined above, having at least one C₁-C₄ alkanol is used, the fraction of the at least one aromatic hydrocarbon in the mixture is at least 50% by weight, preferably at least 70% by weight, particularly preferably at least 80% by weight, in particular at least 90% by weight.

In a preferred embodiment of the variant (i) of the desorption step, the organic solvent used for the desorption comprises at least 80% by weight, preferably at least 90% by weight, particularly preferably at least 95% by weight or more, for example up to 97% by weight, of one or more aromatic hydrocarbons selected from toluene or xylenes, or of a mixture of at least one of these aromatic hydrocarbons with methanol and/or ethanol, wherein the fraction of the at least one aromatic hydrocarbon in the mixture is at least 50% by weight, preferably at least 70% by weight, particularly preferably at least 80% by weight, in particular at least 90% by weight.

In a further preferred embodiment of the variant (i) of the desorption step, the organic solvent used for the desorption comprises exclusively at least one aromatic hydrocarbon.

Preferably, in the variant (i) of the desorption step, the activated carbon is first treated, before the actual desorption, with at least one water-miscible solvent, preferably with at least one C₁-C₄ alkanol, in particular with methanol and/or ethanol, in order to eliminate the water between the activated carbon particles and in the pores situated therein (wash step).

Subsequently thereto, the actual desorption is performed using an organic solvent which comprises a mixture of at least one aromatic hydrocarbon, as defined above, and a C₁-C₄-alkanol, in particular methanol and/or ethanol, wherein the proportion of the at least one aromatic hydrocarbon in the mixture is at least 50% by weight, preferably at least 70% by weight, particularly preferably at least 80% by weight, in particular at least 90% by weight.

As an alternative, or preferably in addition thereto, for the desorption, an organic solvent can be used which comprises at least 80% by weight, preferably at least 90% by weight, particularly preferably 95% by weight or more, for example up to 97% by weight, one or more aromatic hydrocarbons, as defined above.

Likewise preferably, for the further desorption, an organic solvent can also be used which exclusively comprises at least one aromatic hydrocarbon, as defined above.

If the loading of the activated carbon proceeds in an adsorbent arrangement, after the loading and optionally the wash step and/or the treatment with the aqueous acid, an organic solvent, as defined above, is passed through the absorbent arrangement, wherein the bound, optionally neutralized or protonated, valuable materials desorb and elute and at the same time the activated carbon is regenerated. The amount of organic solvent is generally 0.1 to 20 times the amount, in particular 0.5 to 15 times the amount, e.g. 1 to 10-times the amount of the bed volume (BV). The organic solvent (eluent) is generally passed through at a specific flow (specific loading rate) in the range from 0.5 to 20 BV/h, preferably in the range from 0.5 to 10 BV/h, in particular in the range from 1 to 8 BV/h.

With respect to the temperatures during desorption, that stated for loading applies. Elution can be carried either upwardly or downwardly. The elution can take place in the same direction as the loading or in the opposite direction thereto. Preferably, the elution is performed in the opposite direction to loading.

Optionally, before the elution step, the water situated in the pores and between the adsorbent particles, or, if a water-insoluble organic solvent was used for elution, the water-insoluble organic solvent remaining between the adsorbent particles is removed with a water-soluble organic solvent such as methanol and/or ethanol. For this purpose, the water-miscible organic solvent is passed upwardly through the adsorbent arrangement. The amount of water-miscible organic solvent is usually 0.5 to 10 times the amount of the bed volume, in particular 1 to 5 times the amount of the bed volume. The water-miscible organic solvent is preferably passed through at a specific flow rate (specific loading rate) in the range from 0.5 to 10, in particular from 1 to 8, bed volumes per hour.

The elution can be followed by a further wash step in order to remove contaminants optionally present.

The eluate arising in the elution is worked up in a usual manner to obtain the aromatic valuable materials. If the eluate comprises acid, it is generally first removed, for example by an aqueous-extractive workup, or neutralized by adding base separating off the salts formed as a result. Optionally, the eluate can be concentrated in advance, e.g. by removing the solvent in a usual evaporator arrangement. The condensate arising in this case can be reused, for example in a following elution.

The adsorbent arrangement can be operated batchwise and then as one or more, e.g. 2, 3 or 4, series-connected, stationary fixed beds packed with activated carbon. The adsorbent arrangement can also be operated continuously, and then generally has 5 to 50, and in particular 15 to 40, adsorbent beds which can be e.g. components of a “true moving bed” arrangement (see K. Tekeuchi J. Chem. Eng. Jpn., 1978, 11 pp. 216-220), a “Continuous Circulating Annular” (see J. P. Martin, Discuss. Farraday Soc. 1949, p. 7) or a “Simulated Moving Bed” arrangement, as described, for example, in U.S. Pat. No. 2,985,589 and WO 01/72689, and also by G. J. Rossiter et al. Proceedings of AlChE Conference, Los Angeles, Calif., November 1991 or H. J. Van Walsem et al., J. Biochtechnol. 1997, 59, p. 127.

After the desorption according to variant (i), the aromatic valuable materials are generally obtained as eluate in enriched form in the organic solvent used for the desorption.

In variant (ii) of the desorption step, the aromatic valuable materials that are bound to the activated carbon are desorbed by means of steam, by steam-treating the activated carbon that is loaded with the aromatic valuable materials. Preferably, a procedure is followed such that the steam flows through the activated carbon. For this purpose, usually steam is introduced into the bed or fixed bed of activated carbon used for the absorption, for example a column packed with activated carbon. Alternatively, the activated carbon, for this purpose, can also be introduced into a steam stream. In the case of desorption by means of steam, the aromatic valuable materials found on the activated carbon are displaced by the readily absorbable steam and entrained. The activated carbon in this case is regenerated at the same time.

After condensation of the steam, the aromatic valuable materials are obtained in the form of an aqueous solution or suspension. Generally, the aqueous solution or suspension is subjected to a further workup step, in order to separate the desorbed aromatic valuable materials from the aqueous phase.

The aromatic valuable materials which mix only slightly with water can usually be separated via a process of self separation (phase separation). Generally, the product stream, for this purpose, is passed into a phase separator (decanter) where said product stream disaggregates by mechanical settling into two phases (an organic phase and a water phase) which can be taken off separately.

Otherwise, the separation can be used by familiar methods generally known to those skilled in the art for separating aqueous-organic mixtures, such as distillation, liquid extraction or liquid-chromatograph processes.

If the separation may not be achieved, or may be achieved only incompletely in the route of self separation (phase separation), it can also preferably be performed by extraction, using a solvent which is miscible only slightly, or not at all, with water, as defined above.

The desorption by means of steam generally proceeds at ambient pressure or an elevated pressure. Preferably, the desorption by means of steam proceeds at a pressure in the range from 1 to 5 bar, preferably in the range from 1 to 3 bar.

During the desorption by means of steam, the temperature of the activated carbon is usually in the range from 100 to 150° C., preferably in the range from 100 to 130° C.

Usually, the weight ratio of the amount of steam required for the desorption to the amount of the aromatic valuable materials adsorbed on the activated carbon is in the range from 1:1 to 20:1, preferably in the range from 2:1 to 10:1, in particular in the range from 3:1 to 6:1.

In this preferred embodiment of the process according to the invention, the desorption step can proceed after the loading operation and the optionally following wash step can alternatively proceed by means of steam. For this purpose, steam is passed at a continuous flow rate from 0.1 to 0.3 m/s through the absorber arrangement and condensed thereafter. The desorbate is then, to remove the water, worked up by extraction as described above.

In this manner a crude product is obtained which comprises the aromatic valuable materials in enriched form.

The process according to the invention is suitable, in particular, for obtaining aromatic compounds which are formed in the oxidative and/or enzymatic breakdown of lignin, and aromatic compounds which occur naturally in lignin-comprising compositions.

Preferably, the aromatic compounds which can be obtained by this process are selected from compounds of the general formulae (I) and (II)

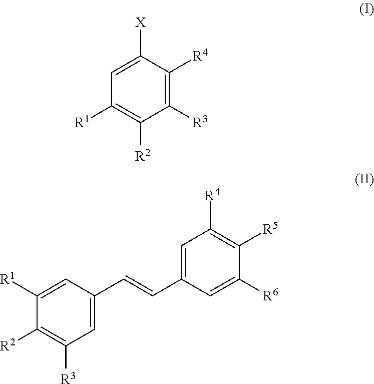

in which
X is H, —CHO, —(C═O)CH₃, —COOH, —CH═CH—COOH or —CH═CH—CH₂—OH and R¹, R², R³, R⁴, R⁵, R⁶, independently of one another are H, —OH, —CH₃ or —OCH₃.

The aromatic compounds of the general formulae (I) are, for example, benzaldehyde and derivatives of benzaldehyde, such as vanillin or isovanillin, acetophenone and derivatives of acetophenone, such as acetovanillone, iso-acetovanillone, orthoacetovanillone, or 3,4,5-trihydroxyacetophenone, anisole, benzcatechin and the methyl ethers therefore such as veratrole or guaiacole, ferulic acid and derivatives of ferulic acid, dehydroconiferyl alcohol, benzoic acid and derivatives of benzoic acid such as vanillin acid, gallic acid and derivatives of gallic acid, such as syringic acid and the like.

The aromatic compounds of the general formulae (II) are, for example, 3,3′-dimethoxy-4,4′-dihydroxystilbene, resveratrol, pinosylvin (3,5-stilbenediol) and the like.

In a preferred embodiment of the process according to the invention, the eluate obtained after the elution or desorption, which eluate comprises the aromatic valuable materials in enriched form, is subjected to a further separation. The separation comprises, for example, a fine distillation, crystallization or a liquid-chromatographic separation. Depending on the nature of the aromatic valuable material mixture and the desired purity, the separation can comprise a plurality of separation steps.

The invention will be explained in more detail with reference to the examples described hereinafter. In this case the examples are not to be taken as limiting for the invention.

In the examples hereinafter, the following abbreviations are used:

BV is bed volume;
DI water is deionized (demineralized) water.

### EXAMPLES

I) Analysis

The content of vanillin, acetovanillon, guaiacol, 3,3′-dimethoxy-4,4′-dihydroxystilbene and other organic components of the aqueous lignin-comprising compositions used was determined by means of high-performance liquid chromatography (HPLC). As stationary phase, the column Chromolith® High Resolution RP18e from Merck (length: 100 mm, diameter 4.6 mm) was used. The analysis temperature was 25° C. In this case two mobile phases were used: HPLC water with 0.1% by weight of 70 percent perchloric acid as mobile phase A; acetonitrile as mobile phase B.

II) Adsorption and Desorption of Aromatic Valuable Materials on Activated Carbon

### Example II.1: Adsorption and Desorption of Aromatic Valuable Materials Such as Vanillin, Acetovanillone, Guaiacol and 3,3′-dimethoxy-4,4′-dihydroxystilbene on Activated Carbon

Activated Carbon Used

In the experiment, the activated carbon Norit® ROY 0.8 from Norit Carbon was used. This activated carbon is a hard-coal-based extrudate and is washed repeatedly with lye (aqueous NaOH) after a steam activation. The bulk density of the activated carbon is 400 g/L. The activated carbon has a moisture content of a max. of 5%.

Lignin-Comprising Composition Used:

The lignin-comprising composition used was black liquor (thin liquor) from wood pulp production. For the experiment, the black liquor was filtered using a metal filter (filter pore size=90 micrometers). The HPLC analysis of the filtered black liquor gave the following concentrations of the organic components: 447 mg/kg of vanillin, 268 mg/kg of acetovanillone, 460 mg/kg of guaiacol and 490 mg/kg of 3,3′-dimethoxy-4,4′-dihydroxystilbene.

Experimental Procedure:

A glass column having an internal diameter of 15 mm and a height of 255 mm was assembled and packed with the activated carbon Norit® ROY 0.8 at a degree of filling of approximately 95%. The bed volume (BV) was approximately 43 mL. The activated carbon was washed with approximately 10 BV of DI water at a rate of approximately 5 BV/h downwardly.

For absorption of the organic components, approximately 12 BV of filtered black liquor was passed through the column at a rate of approximately 2 BV/h downwardly. The column outlet was collected in fractions. The fractions were analyzed for organic components. The loading achieved of 3,3′-dimethoxy-4,4′-dihydroxystilbene (I) was 0.04 mol/L. Thereafter, the activated carbon was washed with approximately 5 BV of DI water at a velocity of approximately 2 BV/h downwardly.

After the wash step, an acid wash was performed in order to protonate the absorbed 3,3′-dimethoxy-4,4′-dihydroxystilbene (I). For this purpose, approximately 1 BV of 5 percent sulfuric acid was passed through the column at a rate of approximately 2 BV/h downwardly. Thereafter, the activated carbon was washed with approximately 5 BV of DI water at a rate of approximately 2 BV/h downwardly.

In order to eliminate the water between the activated carbon particles and in the pores situated therein, after the wash step, approximately 2 BV of pure methanol was passed through the column at a rate of approximately 2 BV/h downwardly.

For desorption of the adsorbed 3,3′-dimethoxy-4,4′-dihydroxystilbene, first approximately 2 BV of a mixture of methanol and toluene in the mass ratio of 9:1 was passed through the column at a rate of 2 BV/h upwardly. For further desorption, then, approximately 3 BV of pure toluene was passed through the column at a rate of 2 BV/h upwardly. In the desorption step, the column outflow was collected in a fraction. This fraction was analyzed for the content of organic components. The degree of desorption achieved of the individual organic components was approximately: 88% for vanillin, 99% for acetovanillone, 83% for guaiacol and 3% for 3,3′-dimethoxy-4,4′-dihydroxystilbene.

After the desorption step, approximately 1 BV of pure methanol was passed through the column at a rate of approximately 2 BV/h upwardly.

After the methanol scrubbing, the activated carbon was washed with approximately 10 BV of DI water at a rate of approximately 5 BV/h upwardly.

All process steps were carried out at room temperature.

### Example II.2: Adsorption and Desorption of Aromatic Valuable Materials Such as Vanillin, Acetovanillon, Guaiacol and 3,3′-dimethoxy-4,4′-dihydroxystilbene on Activated Carbon

Activated Carbon Used:

In the experiment, the activated carbon Aquacarb™ 207C from Chemviron Carbon was used. This activated carbon is a coconut-based granulated activated carbon activated with steam. The bulk density of the activated carbon is 450 g/L. The activated carbon has a moisture content of a max. of 5%.

Lignin-Comprising Composition Used:

The lignin-comprising composition used was black liquor (thin liquor) from wood pulp production. For the experiment, the black liquor was filtered using a metal filter (filter pore size=90 micrometers). The HPLC analysis of the filtered black liquor gave the following concentrations of the organic components: 457 mg/kg of vanillin, 349 mg/kg of acetovanillone, 506 mg/kg of guaiacol and 308 mg/kg of 3,3′-dimethoxy-4,4′-dihydroxystilbene.

Experimental Procedure:

A glass column having an internal diameter of 15 mm and a height of 255 mm was assembled and packed with the activated carbon Aquacarb™ 207C at a degree of filling of approximately 95%. The bed volume (BV) was approximately 43 mL. The activated carbon was first washed with approximately 10 BV of DI water at a rate of approximately 5 BV/h downwardly.

For adsorption of the organic components, approximately 12 BV of filtered black liquor was passed through the column at a rate of approximately 2 BV/h downwardly. The column outlet was collected in fractions. The fractions were analyzed for organic components. The loading achieved of the individual organic components on the activated carbon was: 0.02 mol/L vanillin, 0.01 mol/L acetovanillone, 0.03 mol/L guaiacol and 0.01 mol/L 3,3′-dimethoxy-4,4′-dihydroxystilbene. Thereafter, the activated carbon was washed with approximately 5 BV of DI water at a velocity of approximately 2 BV/h downwardly.

After the wash step, an acid wash was performed in order to protonate the adsorbed organic anions. For this purpose, approximately 1 BV of 5 percent sulfuric acid was passed through the column at a rate of approximately 2 BV/h downwardly. Thereafter, the activated carbon was washed with approximately 5 BV of DI water at a rate of approximately 2 BV/h downwardly.

In order to eliminate the water between the activated carbon particles and the pores situated therein, after the wash step, approximately 2 BV of pure methanol was passed through the column at a rate of approximately 2 BV/h downwardly.

For desorption of the adsorbed organic components, first approximately 2 BV of a mixture of methanol and toluene in the mass ratio of 1:1 was passed through the column at a rate of 2 BV/h upwardly. For further desorption, then, approximately 3 BV of pure toluene was passed through the column at a rate of 2 BV/h upwardly. In the desorption step, the column outflow was collected in a fraction. This fraction was analyzed for the content of organic components. The degree of desorption achieved of the individual organic components was: 89% for vanillin, 95% for acetovanillone, 89% for guaiacol and 8% for 3,3′-dimethoxy-4,4′-dihydroxystilbene.

After the desorption step, approximately 1 BV of pure methanol was passed through the column at a rate of approximately 2 BV/h upwardly.

After the methanol scrubbing, the activated carbon was washed with approximately 10 BV of DI water at a rate of approximately 5 BV/h upwardly.

All process steps were carried out at room temperature.

### Example II.3: Adsorption and Desorption of Vanillin on Activated Carbon

Activated Carbon Used:

In the experiment, the activated carbon Norit® ROY 0.8 from Norit was used. This activated carbon is a hard-coal-based extrudate and is washed repeatedly with lye (aqueous NaOH) after a steam activation. The bulk density of the activated carbon is 400 g/L. The activated carbon has a moisture content of approximately 5%.

Valuable Material-Comprising Composition Used:

As valuable material-comprising composition, a solution of 0.1 M sodium hydroxide solution and vanillin was used. The HPLC analysis of the solution gave a content of vanillin of 2834 mg/kg.

Experimental Procedure:

A glass column having an internal diameter 30 mm and a height of 1000 mm was assembled and packed with the activated carbon Norit® ROY 0.8 at approximately 90% degree of filling.

The bed volume (BV) was approximately 636 mL. The activated carbon was next washed with approximately 10 BV of DI water at a rate of approximately 5 BV/h downwardly.

For adsorption of the vanillin, at room temperature, approximately 20 BV of the vanillin-comprising solution was passed through the column downwardly at a rate of approximately 4 BV/h. The column outflow was collected in fractions. The fractions were analyzed for organic components. The loading of vanillin achieved on the activated carbon was approximately 0.32 mol/L. Thereafter, the activated carbon was washed downwardly at room temperature with approximately 5 BV of DI water at a rate of approximately 2 BV/h.

After the wash step, an acid wash was performed in order to protonate the adsorbed vanillate anions. For this purpose, at room temperature, approximately 1 BV of 5 percent sulfuric acid were passed through the column downwardly at a rate of approximately 2 BV/h. Thereafter, likewise at room temperature, the activated carbon was washed downwardly with approximately 5 BV of DI water at a rate of approximately 2 BV/h.

The adsorbed vanillin was desorbed by means of steam. In this case, at a mass flow rate of approximately 300-500 g/h, water was vaporized via a falling-film evaporator (at approximately 140-150° C.) and continuously passed through the column. In this procedure the pressure in the column was 1.013 bar and the temperature was approximately 100-120° C. Then, the steam was condensed and collected in a fraction. Vanillin was detectable in this fraction.

1.-13. (canceled)

14. A process for obtaining aromatic compounds from aqueous alkaline lignin-comprising compositions that have a pH of at least pH 10, which comprises treating the aqueous alkaline lignin-comprising composition with activated carbon, separating off the activated carbon from the aqueous alkaline lignin-comprising composition and then subjecting the activated carbon to a desorption step for obtaining the aromatic compounds, wherein the desorption step comprises treating the activated carbon
(i) with an organic solvent which consists essentially of one or more aromatic hydrocarbons, or is a mixture of at least one aromatic hydrocarbon with at least one C₁-C₄ alkanol, or
(ii) with steam,
wherein an eluate that comprises the aromatic compounds is obtained.

15. The process according to claim 14, wherein, after the aqueous alkaline lignin-comprising composition is separated off, the activated carbon is first treated with an aqueous mineral acid solution and then with an organic solvent.

16. The process according to claim 14, wherein the organic solvent is at least one aromatic hydrocarbon.

17. The process according to claim 16, wherein the aromatic hydrocarbon is selected from the group consisting of toluene and xylenes.

18. The process according to claim 14, wherein the aqueous alkaline lignin-comprising composition is passed through a bed of activated carbon for treatment thereof.

19. The process according to claim 14, wherein the activated carbon is an activated carbon that is activated with steam.

20. The process according to claim 14, wherein the activated carbon has a specific surface area in the range from 500 to 1500 m²/g, determined by nitrogen adsorption by the BET method as specified in DIN ISO 9277:2003-05.

21. The process according to claim 14, wherein the activated carbon has an adsorption capacity of at least 15 g of methylene blue per 100 g of activated carbon.

22. The process according to claim 14, wherein the activated carbon has a grain size in the range from 0.2 to 5 mm.

23. The process according to claim 14, wherein the eluate is subjected to a separation of the aromatic compounds present therein.

24. The process according to claim 14, wherein the aqueous alkaline lignin-comprising composition is a black liquor.

25. The process according to claim 14, wherein the aromatic compounds are selected from the group consisting of aromatic compounds which are formed in the oxidative and/or enzymatic breakdown of lignin, and aromatic compounds which occur naturally in lignin-comprising compositions.

26. The process according to claim 14, wherein the aromatic compounds are selected from the group consisting of compounds of the general formulae (I) and (II) 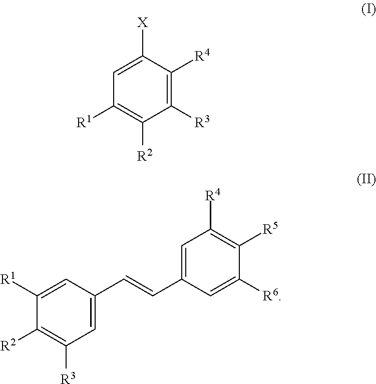
in which
X is H, —CHO, —(C═O)CH₃, —COOH, —CH═CH—COOH or —CH═CH—CH₂—OH and R¹, R², R³, R⁴, R⁵, R⁶, independently of one another are H, —OH, —CH₃ or —OCH₃.
